(19) United States
(12) Patent Application Publication
(10) Pub. No.: US 2022/0351831 A1
(43) Pub. Date: Nov. 3, 2022

(54) CUSTOMIZED FOOD LABELS FOR PREFERENTIAL INSTRUCTION

(71) Applicant: International Business Machines Corporation, Armonk, NY (US)

(72) Inventors: Venkata Vara Prasad Karri, Visakhapatnam (IN); Partho Ghosh, Kolkata (IN); Hemant Kumar Sivaswamy, Pune (IN); Sri Harsha Varada, Vizianagaram (IN)

(21) Appl. No.: 17/306,086

(22) Filed: May 3, 2021

(51) Int. Cl. G16H 20/60; G16H 10/60; G06F 16/9035

(52) U.S. Cl. CPC G16H 20/60; G16H 10/60; G06F 16/9035

(57) ABSTRACT

In a method for labeling nutritional parameters a processor receives a food order. The food order a first user profile identifying user preferences. A processor tracks preparation specifics of the food order. A processor determines a custom label for the food order based on the first user profile and the preparation specifics.

### BACKGROUND

The present invention relates generally to the field of food preparation and labeling, and more particularly to customizing a food label based on user-specific and dish-specific information.

The nutrition facts label (also known as the nutrition information panel, and other slight variations) is a label required on most packaged food in many countries, showing what nutrients are in the food. The nutrition facts label may include recommended daily amounts or recommended daily limits for nutrients. Labels are usually based on official general nutritional rating systems. Most countries also release overall nutrition guides for general educational purposes. Nutrition facts labels are one of many types of food labels required by regulation or applied by manufacturers. With increases in knowledge about food and nutrition, many people are becoming increasingly conscious on the kind of food they are consuming, the ingredients, and the calorific value of the food.

### SUMMARY

According to one embodiment of the present invention, a computer-implemented method, a computer program product, and a computer system are provided. A processor receives a food order. The food order may include a first user profile identifying user preferences. A processor tracks preparation specifics of the food order. A processor determines a custom label for the food order based on the first user profile and the preparation specifics.

### BRIEF DESCRIPTION OF THE DRAWINGS

FIG. 1 is a functional block diagram illustrating a custom food labeling environment, in accordance with an embodiment of the present invention;

FIG. 2 is a flowchart depicting operational steps of a food preparation program, in accordance with an embodiment of the present invention; and

FIG. 3 is a schematic diagram depicting a flow of a custom food labeling environment, in accordance with an embodiment of the present invention; and

FIG. 4 is a block diagram of components of a computing device in accordance with one illustrative embodiment of the present invention.

### DETAILED DESCRIPTION

In many jurisdictions around the world, the food service industry is governed by regulations and standards, such as required nutrition facts labels, for packaged food distributed in stores. Other food service providers (such ‘organized’ providers such as restaurants; or ‘unorganized’ provides such as street vendors), are not required to label food with nutrition information, even if some menus may contain information regarding food allergies or compliance with dietary restrictions. Embodiments disclosed herein, on the other hand, recognize that there can be a need for some customers to be able to customize, and be informed about, individual dishes they order from food service providers. Furthermore, by compiling a user profile and tracking the preparation specifics of individual dishes, the embodiments herein address the need to label food so that consumers know the content in the specific dishes they are consuming.

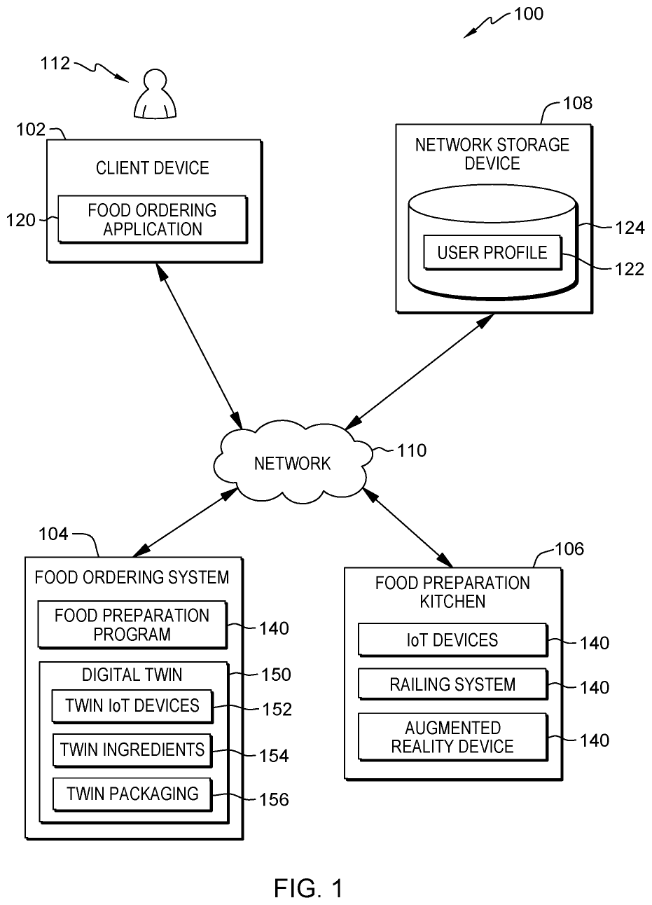

Turning now to the figures, FIG. 1 is a functional block diagram illustrating a digital twin food preparation environment 100, in accordance with an embodiment of the present invention. The environment 100 includes a client device 102, a food ordering system 104, a food preparation kitchen 106, and a network storage device 108. Additional devices, such as additional client devices 102, may be included in the environment 100 without diverging from the disclosed embodiments of the invention. In certain embodiments, as illustrated, the client device 102, the food ordering system 104, the food preparation kitchen 106, and the network storage device 108 are communicatively coupled via a communication network 110. The communication network 110 may be a local area network (LAN), a wide area network (WAN) such as the Internet, any combination thereof, or any combination of connections and protocols that will support communications between the client device 102, the food ordering system 104, the food preparation kitchen 106, and the network storage device 108 in accordance with embodiments of the invention. The communication network 110 may include wired, wireless, or fiber optic connections. In certain embodiments, the client device 102, the food ordering system 104, and the network storage device 108 may communicate without requiring the communication network 110, instead communicating via one or more dedicated wire connection or other forms of wired and wireless electronic communication.

A user 112 may access the environment 100, to place a food order, through the client device 102 running a food ordering application 120. The client device 102 may include, for example, a smartphone, a computer, augmented reality (AR) glasses or smart lens, or other consumer device; or the client device 102 may include dedicated a multiple-user device such as a kiosk. The food ordering application 120 may collect a user profile 122 from the user 112 so that placing a food order through the food ordering application 120 automatically populates user preferences. The user preferences may include, for example, body metrics such as height, weight, body-fat composition, or other measurements that the user 112 considers potentially relevant to food choices. The user profile 122 may also include user preferences specifying desired measurements for the body metrics, such that the ordering application 120 may designate recommendations for restrictions on caloric intake and/or nutritional replacements for ingredients within the food order. The user preferences may also include medical conditions such as food allergies or current prescriptions, so that the food ordering application 120 automatically adjusts a food order to avoid products that may cause an adverse reaction to the user, or alert the user 112 with a warning before the food order is completed. The user preferences may also include dietary preferences such as vegetarian, carnivore, non-alcoholic, sugar-free, low-fat, high-protein, etc. The user preferences may also include flavor preferences such as a level of spiciness or a preferences for the taste of one type of protein over another.

In certain embodiments, the user profile 122 may be connected with health monitoring devices such as wearable heart monitors, step counters, or exercise equipment. The user preferences may thus include dynamically changing adjustments to energy or other nutritional requirements for the user 112, based on the tracked activity of the user 112. Further adjustment to the user profile 122 and the user preferences may be accomplished at any time through the client device 102, or by logging in to the user profile 122 through access to the communication network 110. That is, the user profile 122 may be stored locally on the client device 102, or may be stored (as illustrated) on a data storage 124 connected to the client device 102 through the communication network 110.

The data storage 124 may include read-only memory (“ROM”), random access memory (“RAM”) (e.g., dynamic RAM (“DRAM”), synchronous DRAM (“SDRAM”), and the like), electrically erasable programmable read-only memory (“EEPROM”), flash memory, a hard disk, a secure digital (“SD”) card, other suitable memory devices, or a combination thereof. The data storage 124 is illustrated food ordering system 104 food preparation kitchen 106 as part of the network storage device 108, but the data storage 124 may be incorporated with any or each of the client device 102, the food ordering system 104, or the food preparation kitchen 106.

With the user profile 122 stored within the environment 100, the food ordering application 120 may next begin taking a food order. The user 112 may select one or multiple user profiles 122 within the food ordering application 120, depending on who will be consuming the food order. For example, if a family is going to eat the food order together, each of the family member's user profiles will be included in the ordering process. The user 112 then selects one or more dishes from a menu within the food ordering application 120. Each dish may be associated with a specific user profile 122, or a selected dish may be assigned to a collection of the user profiles 122 (e.g., the group of user profiles 122 as a whole). When the user 112 finishes adding dishes to the food order, the food ordering application 120 may automatically adjust the dishes and/or the food order based on the user profiles 122. For example, the food order may be adjusted to substitute ingredients based on the user preferences in the user profile 122. The food ordering application 120 may also be enabled to adjust/substitute ingredients to maintain a food taste. The food ordering application 120 then sends the food order through the communication network 110, where it may be stored on the network storage device 108.

The food ordering system 104 communicates with the communication network 110 and may run a food preparation program 140 based on information from the communication network 110.

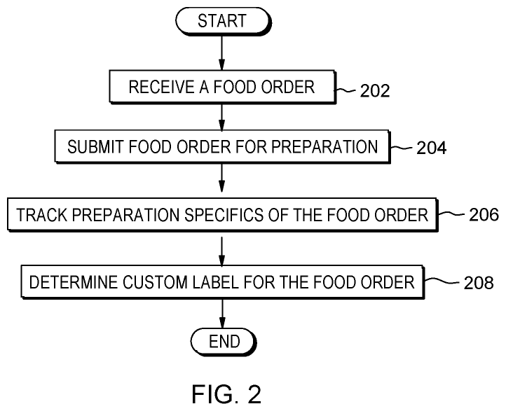

FIG. 2 is a flowchart depicting operational steps of the food preparation program 140, in accordance with an embodiment of the present invention. The food preparation program 140 receives a food order (block 202). Typically, the food order is received from the food ordering application 120 through the communication network 110. Additionally or alternatively, the food order may be received from a worker (e.g., restaurant employee, telephone ordering service) inputting the food order directly into the food ordering system 104. The food order may include one or more user profiles 122, which may also be received from the communication network 110 or through direct input into the food ordering system 104.

If the food order was not customized to the user profile 122 by the food ordering application 120, the food preparation program 140 may adjust the food order by providing substitutes, recommending a replacement ingredient for a food item in the food order that does not meet the user preferences, and/or changing amounts of ingredients as described above. Additionally or alternatively, the food preparation program 140 may secondarily adjust the food order based on availability of ingredients from the food preparation kitchen 106.

The food preparation program 140 may then submit the food order (with any alterations configured to the user profile 122) for preparation by the food preparation kitchen (block 204). The food preparation kitchen 106 may be included as part of any individuals or businesses such as restaurants, street vendors, hotels, factories, cafeterias, or other food preparers, who prepare the food order according to the instructions included by the food ordering system 104. In certain embodiments, the food preparation kitchen 106 may include an auto packing railing system 144 configured to prepare the food order. The auto packing railing system 144 uses a tray passing along a railing through various depositing areas to prepare the food order. At each depositing area, the auto packing railing system 144 deposits a type of food before the tray moves passed. For example, pumps may convey sauces to a sauce depositing area, or belts may convey meat portions, vegetables, and pasta to depositing areas in large trays. The food preparation kitchen 106 then uses depositing machines to weigh and place the required amounts of a given component directly onto the tray. Frozen items may be fed into a depositor with a rotating bottom containing several cavities that accept a designated amount of a food material. The cavities may be adjustable in size to measure the designated amount of food material to the granularity of an ounce or a gram. The cavities then open directly over a designated slot of the tray or the bag. After the meat or vegetable is deposited on the tray, the sauce may be dispensed to coat the desired meal component.

The auto packing railing system 144 may enable the food preparation kitchen 106 to prepare many food orders on a more commercial scale, but the food preparation kitchen 106 may also include TOT enabled devices 142 or augmented reality devices 146 to keep track of food orders prepared individually using a digital twin 150, as explained in detail below.

The food preparation program 140 may then track preparation specifics of the food order using a digital twin of the food preparation kitchen 106 (block 206). The digital twin of the food preparation kitchen 106 may include twin IoT devices 152, twin ingredients 154, and/or twin packaging 156. The preparation specifics may include amounts and types of ingredients, times for cooking and/or baking, temperatures, types of instruments or utensils used to cook, or other specifics. The preparation specifics may be tracked using a variety of potential tracking methods. The twin IoT devices 152 digitally represent the IoT devices 142 that are physically used in preparing the food order. For example, the IoT devices 142 may include jars and spoons which would have the capability to dynamically identify the ingredients added to the food order, and the amounts of the ingredients. The IoT devices 142 may also include thermometers that convey a temperature to the digital twin 150. The digital twin 150 may also be given the timing of vegetable peeling or cutting, the amount of water that the food was cooked in, the size of pieces during cooking, and the pH level of water during cooking, as these actions can change the amount of nutrients (particularly vitamin C) that remains in the food after cooking. The TOT enabled devices 142 thus measure quantities, cooking duration, cooking temperatures, and other preparation specifics directly, such that the digital twin 150 is able to digitally track each aspect that may affect nutritional value for all individual dishes. These aspects may be compared with artificial intelligence models for nutritional value, and data that shows medical and health benefits. Furthermore, the digital twin 150 enables monitors and models these health benefits without the need for input from people while they are preparing the food order.

When the food order has been completed and the preparation specifics are tracked, the food preparation program 140 may generate a custom label for the food order (block 208). The custom label may be based on the user profile 122 and the preparation specifics such that the user 112 may be able to determine accurate nutritional information directly related to the nutritional desires that the user 112 has input into the user profile 122. For example, the custom label may include typical caloric and macronutrient breakdown of the food order, but may display a protein content more prominently due to a user preference indicating protein consumption is a priority. Additionally or alternatively, the custom label may leave off certain vitamins or minerals for a user profile 122 that indicates no interest in that vitamin or mineral.

In further contrast to a typical nutrition facts label, the custom label may include the measured values for the information as measured during the preparation process. That is, the custom label may include a breakdown of a plurality of user profiles 122 that may be sharing the food order. Specifically, if multiple user profiles 122 are associated with a particular dish in the food order, the custom label may include a recommended portion breakdown for each of the user profiles 122. The portions may be determined based on the nutritional needs or desires that have been recorded as user preferences within the user profiles 122. The user 112 may utilize smart devices, such as augmented reality, to enable proper distribution between the portions associated with the various user profiles 122.

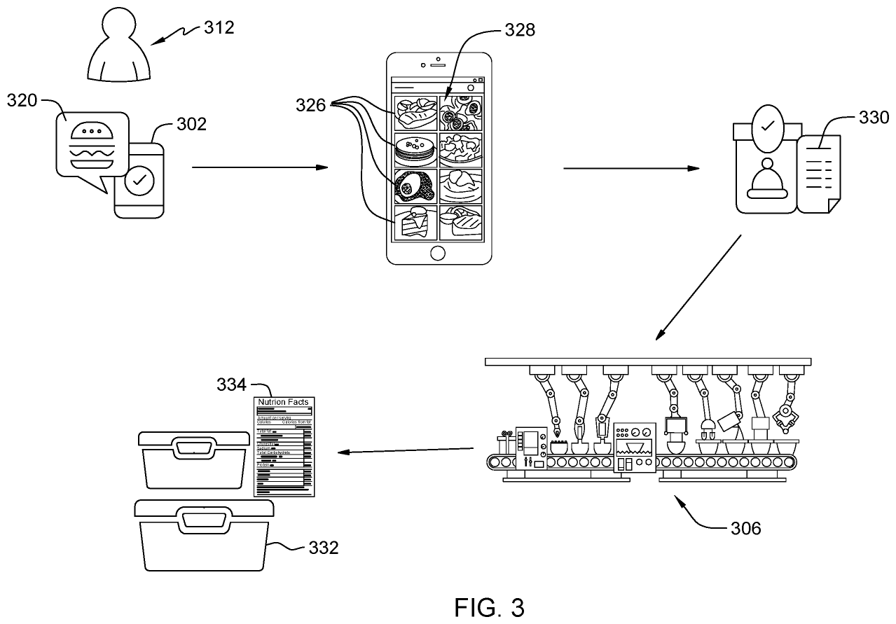

FIG. 3 is a schematic diagram depicting a flow of a custom food labeling environment, in accordance with an embodiment of the present invention. In a first illustrated step, a user 312 opens a food ordering application 320 on a client device 302. The food ordering application 320 may invite the user 312 to select a user profile (or multiple user profiles) for any food order that will be submitted. Then the user 312 chooses from available dishes 326 on a menu 328 within the food ordering application 320. The food ordering application 320 may customize the available dishes 326 based on the user profiles associated with the client device and/or selected when the food ordering application 320 was opened. When the dishes 326 are selected, the user places a food order 330, which the food ordering application 330 uploads to a communication network. The food order 330 is then received by a food preparation program associated with a food service provider 306. The food service provider 306 prepares the food 332, as described in detail above. The food preparation program then determines a label 334, which is printed and attached to the food 332.

When the food arrives, or is picked up by the user 312, the user 312 may use augmented reality or a camera scan of the label 334 to generate a more comprehensive analysis of the food 332 than is available on the label 334 alone. For example, while the label 334 may show an amount of a nutrient (e.g., vitamin C), the scan of the label 334 may bring up a report on the food ordering application 320 that details the preparation specifics that contributed to the amount of the nutrient (e.g., cooking time, amount of cooking water, pH of the water). Furthermore, augmented reality of the size of the food 332 may inform the user 312 with an updated portion size for all the people that will consume the food 332. For example, the food order may have been submitted with a single user profile, but upon arrival will be shared by multiple people. The augmented reality scan of the food can enable the user 312 to divide the food 332 into portions for each person based on the profiles that are shared with the user 312 wearing the augmented reality or using the camera scan.

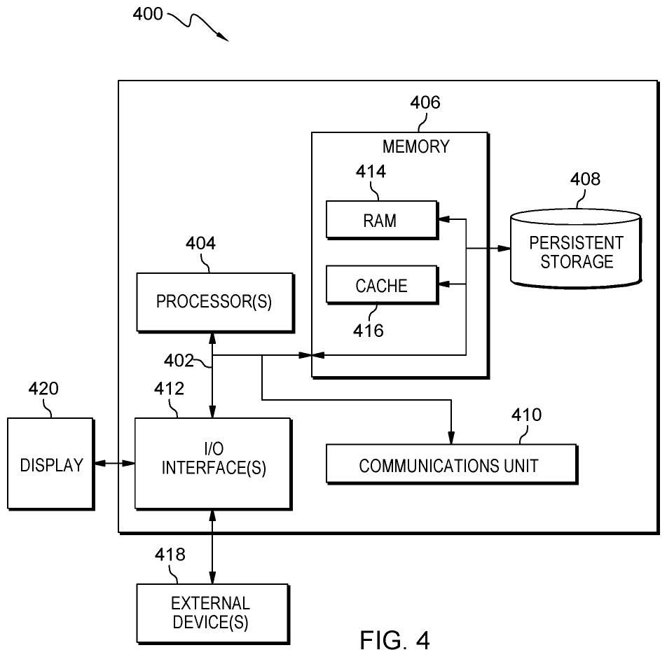

FIG. 4 depicts a block diagram of components of a computing device 400 in accordance with an illustrative embodiment of the present invention. The computing device 400 may represent any of the devices (e.g., the client device 102, the food ordering system 104, the food preparation kitchen 106, and the network storage device 108) described above, or a combination of the devices, in the embodiments where the devices are embodied as components of a single computing device. It should be appreciated that FIG. 4 provides only an illustration of one implementation and does not imply any limitations with regard to the environments in which different embodiments may be implemented. Many modifications to the depicted environment may be made.

The computing device 400 includes communications fabric 402, which provides communications between RAM 414, cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

The software components (e.g., food ordering application, food preparation program, user profiles) may be stored in persistent storage 408 and in memory 406 for execution and/or access by one or more of the respective computer processors 404 via cache 416. In an embodiment, persistent storage 408 includes a magnetic hard disk drive. Alternatively, or in addition to a magnetic hard disk drive, persistent storage 408 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. The proxy, application, access manager, collection page, authentication tool, or multi-factor authentication page may be downloaded to persistent storage 408 through communications unit 410.

I/O interface(s) 412 allows for input and output of data with other devices that may be connected to the computing device 400. For example, I/O interface 412 may provide a connection to external devices 418 such as a keyboard, keypad, a touch screen, and/or some other suitable input device. External devices 418 can also include portable computer readable storage media such as, for example, thumb drives, portable optical or magnetic disks, and memory cards. Software and data used to practice embodiments of the present invention (e.g., proxy, application, access manager, collection page, authentication tool, or multi-factor authentication page) can be stored on such portable computer readable storage media and can be loaded onto persistent storage 408 via I/O interface(s) 412. I/O interface(s) 412 also connect to a display 420.

Display 420 provides a mechanism to display data to a user and may be, for example, a computer monitor.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method for labeling nutritional parameters, comprising:
receiving, by one or more processors, a food order, wherein the food order comprises a first user profile identifying user preferences;
submitting the food order for preparation by a food preparation kitchen;
tracking preparation specifics of the food order using a digital twin of the food preparation kitchen; and
generating a custom label for the food order based on the first user profile and the preparation specifics.

2. The method of claim 1, wherein the user preferences comprise a selection from the group consisting of body metrics, medical conditions, food allergies, dietary preferences, and caloric preferences.

3. The method of claim 1, wherein the food order comprises a second user profile.

4. The method of claim 3, wherein the custom label comprises a first portion allotment for the first user profile and a second portion allotment for the second user profile.

5. The method of claim 1, wherein the digital twin comprises a selection from the group consisting of: an internet-of-things (IOT) enabled device twin, a food processing twin, a food packaging twin, and an ingredients twin.

6. The method of claim 1, wherein the preparation specifics comprise volumetric measurements of ingredients determined by analyzing images captured by a camera.

7. The method of claim 1, comprising determining, with an augmented reality device, that a divided portion of the food order matches a portion size specified on the custom label.

8. The method of claim 1, comprising recommending a replacement ingredient for a food item in the food order that does not meet the user preferences of the first user profile.

9. The method of claim 8, wherein the custom label identifies the replacement ingredient.

10. A computer program product comprising:
one or more computer readable storage media, and program instructions collectively stored on the one or more computer readable storage media, the program instructions comprising:
program instructions to receive a food order, wherein the food order comprises a first user profile identifying user preferences;
program instructions to submit the food order for preparation by a food preparation kitchen;
program instruction to track preparation specifics of the food order using a digital twin of the food preparation kitchen; and
program instructions to generate a custom label for the food order based on the first user profile and the preparation specifics.

11. The computer program product of claim 10, wherein the user preferences comprise a selection from the group consisting of body metrics, medical conditions, food allergies, dietary preferences, and caloric preferences.

12. The computer program product of claim 10, wherein the digital twin comprises a selection from the group consisting of: an internet-of-things (TOT) enabled device twin, a food packaging twin, and an ingredients twin.

13. The computer program product of claim 10, wherein the preparation specifics comprise volumetric measurements of ingredients determined by analyzing images captured by a camera.

14. The computer program product of claim 10, comprising program instructions to recommend a replacement ingredient for a food item in the food order that does not meet the user preferences of the first user profile.

15. A computer system comprising:
one or more computer processors, one or more computer readable storage media, and program instructions collectively stored on the one or more computer readable storage media for execution by at least one of the one or more computer processors, the program instructions comprising:
program instructions to receive a food order, wherein the food order comprises a first user profile identifying user preferences;
program instructions to submit the food order for preparation by a food preparation kitchen;
program instruction to track preparation specifics of the food order using a digital twin of the food preparation kitchen; and
program instructions to generate a custom label for the food order based on the first user profile and the preparation specifics.

16. The computer system of claim 15, wherein the user preferences comprise a selection from the group consisting of body metrics, medical conditions, food allergies, dietary preferences, and caloric preferences.

17. The computer system of claim 15, wherein the preparation specifics comprise measurements taken by internet-of-things (TOT) enabled devices connected to a wifi network.

18. The computer system of claim 15, wherein the preparation specifics comprise measurements captured by a camera.

19. The computer system of claim 15, comprising recommending a replacement ingredient for a food item in the food order that does not meet the user preferences of the first user profile.

20. The computer system of claim 15, comprising program instructions to recommend a replacement ingredient for a food item in the food order that does not meet the user preferences of the first user profile.
